(19) United States
(12) Patent Application Publication
(10) Pub. No.: US 2023/0279448 A1
(43) Pub. Date: Sep. 7, 2023

(54) METHOD AND PROCESS ARRANGEMENT FOR IMPROVING A SEPARATION AND SOLID PRODUCT

(71) Applicant: UPM-KYMMENE CORPORATION, Helsinki (FI)

(72) Inventors: Mauno Miettinen, Lappeenranta (FI); Juha Tamper, Levanen (FI); Sami Turunen, Lappeenranta (FI); Meri Ventola, Leipzig (DE)

(21) Appl. No.: 18/009,064

(22) PCT Filed: Jun. 7, 2021
(86) PCT No.: PCT/FI2021/050421

§ 371 Date: Dec. 8, 2022

(30) Foreign Application Priority Data
Jun. 12, 2020 (FI) 20205612

(51) Int. Cl. C12P 7/22; C08L 97/00; C08H 8/00; B01D 21/01; B01D 37/03; B01D 36/04; B01D 21/24

(52) U.S. Cl. CPC C12P 7/22; C08L 97/005; C08H 8/00; B01D 21/01; B01D 37/03; B01D 36/04; B01D 21/2488

(57) ABSTRACT

A method and a process arrangement for improving a solid-liquid separation of solids from a hydrolyzed material formed in an enzymatic hydrolysis of lignocellulosic material, comprising following step: i) adding a flocculant additive to the hydrolyzed material or to a stream separated from the hydrolyzed material to form a mixture, and ii) separating a solid stream and a liquid stream from the mixture by means of a decantation or gravitational separation. Further is disclosed a solid product.

### TECHNICAL FIELD

The present disclosure relates to a method for improving a solid-liquid separation of solids. The present disclosure further relates to a process arrangement for improving a solid-liquid separation of solids. The present disclosure further relates to a solid product.

### BACKGROUND

Different methods are known for converting bio-based raw material, such as wood-derived raw material, into various products. Further, different methods are known to separate product fractions from each other.

### SUMMARY

The method and process arrangement and solid product are characterized by what are presented in the claims.

The method for improving a solid-liquid separation of solids from a hydrolyzed material formed in an enzymatic hydrolysis of lignocellulosic material is disclosed. The method comprises adding a flocculant additive to the hydrolyzed material or to a stream separated from the hydrolyzed material to form a mixture, and separating a solid stream and a liquid stream from the mixture by means of a decantation or gravitational separation.

Further is disclosed the process arrangement. The process arrangement comprises at least one feeder for feeding a flocculant additive to the hydrolyzed material or to a stream separated from the hydrolyzed material to form a mixture, and at least one decanter or gravitational separator for separating a solid stream and a liquid stream from the mixture.

Further is disclosed a solid product formed by the method.

### BRIEF DESCRIPTION OF THE DRAWINGS

The accompanying drawings, which are included to provide further understanding of the embodiments and constitute a part of this specification, illustrates embodiments. In the drawings:

FIG. 1 presents a flow chart of one embodiment of the method for separating solids,

FIG. 2 presents a flow chart of another embodiment of the method for separating solids,

FIG. 3 presents a flow chart of another embodiment of the method for separating solids,

FIG. 4 presents a flow chart of another embodiment of the method for separating solids, and

FIG. 5 presents a flow chart of another embodiment of the method for separating solids.

### DETAILED DESCRIPTION

In a method for improving a solid-liquid separation of solids from a hydrolyzed material formed in an enzymatic hydrolysis of lignocellulosic material, the method comprises following steps
(i) adding a flocculant additive to the hydrolyzed material or to a stream separated from the hydrolyzed material to form a mixture, and
ii) separating a solid stream and a liquid stream from the mixture by means of a decantation or gravitational separation.

A process arrangement for improving a solid-liquid separation of solids from a hydrolyzed material formed in an enzymatic hydrolysis of lignocellulosic material may comprise at least one feeder for feeding a flocculant additive to the hydrolyzed material or to a stream separated from the hydrolyzed material to form a mixture and at least one decanter or gravitational separator for separating a solid stream and a liquid stream from the mixture in order to treat at least the part of the hydrolyzed material after the enzymatic hydrolysis.

In one embodiment, the method comprises at least one enzymatic hydrolysis step, such as a first enzymatic hydrolysis step. In one embodiment, the process arrangement comprises at least one enzymatic hydrolysis equipment, e.g. in the first enzymatic hydrolysis step.

In the method and process arrangement, solids may be recovered. In this context, the solids mean any solid material after a last separation step, for example the solid stream from the last decantation or gravitational separation step or a solid product fraction from a last filtration step. In the method and process arrangement, at solid-liquid separation least one separation step is performed to separate the solids, such as the solid stream and/or the solid product fraction. The last separation step may be decantation, gravitational separation, solid-liquid separation or other separation. The solids comprise at least lignin. In one embodiment, the solids consist of the lignin. A solid product may be formed from the solids. The solid product contains at least lignin.

In this context, the lignocellulosic material means a feed which is subjected to an enzymatic hydrolysis, such as to a first enzymatic hydrolysis step. In one embodiment, the lignocellulosic material is a feed comprising solid cellulose particles or a fraction comprising solid cellulose particles.

In this context, the hydrolyzed material is a product stream from the enzymatic hydrolysis or a selected fraction of the product stream. The hydrolyzed material may be taken from the first enzymatic hydrolysis step or the second enzymatic hydrolysis step or any further enzymatic hydrolysis step.

In this context, the additive means any additive, auxiliary material or the like. The additive may be a flocculant additive or solid additive.

In this context, the decantation may mean any decantation, desilting, elutriation or the like method. The decantation may be carried out in a decanter, decanter centrifuge, solid bowl centrifuge type separator or another suitable decantation device. In this context, the decanter may mean any decanter, decanter centrifuge, solid bowl centrifuge type separator or another suitable decantation device. In this context, the gravitational separation may mean any gravitational method, or gravity-based settling or other gravity-based method. The gravitational separation can be carried out in a gravity separator, centrifugal device or another suitable gravitational separation device. In this context, the gravitational separator may mean any gravitational separator, gravity separator, centrifugal device or another suitable gravitational separation device. A flocculation may be utilized in the decantation and gravitational separation.

The method as disclosed in the current specification may comprise providing the lignocellulosic material, e.g. a fraction comprising solid cellulose particles. The lignocellulosic material may be provided starting e.g. from a wood-based feedstock originating from wood-based raw material and comprising wood chips, that is subjected to at least one pretreatment to form a liquid fraction and a fraction comprising solid cellulose particles. By the expression “pretreating” or “pretreatment” should be understood in this specification, unless otherwise stated, (a) process(es) conducted to convert wood-based feedstock to a fraction comprising solid cellulose particles. As a result of the pretreatment, in addition to the fraction comprising solid cellulose particles and lignin, a liquid fraction may be formed. The liquid fraction may be separated from the fraction comprising solid cellulose particles and lignin. The fraction comprising solid cellulose particles may further include an amount of lignocellulose particles as well as lignin particles in free form. The fraction comprises lignin chemically bonded to the cellulose particles.

The wood-based raw material may be selected from a group consisting of hardwood, softwood, and their combination. The wood-based raw material may e.g. originate from pine, poplar, beech, aspen, spruce, eucalyptus, ash, or birch. The wood-based raw material may also be any combination or mixture of these. The wood-based raw material may be broadleaf wood. Preferably the wood-based raw material is broadleaf wood due to its relatively high inherent sugar content, but the use of other kinds of wood is not excluded. The broadleaf wood may be selected from a group consisting of beech, birch, ash, oak, maple, chestnut, willow, poplar, and any combination of mixture thereof.

Providing the wood-based feedstock may comprise subjecting wood-based raw material to a mechanical treatment selected from debarking, chipping, dividing, cutting, beating, grinding, crushing, splitting, screening, and/or washing the wood-based raw material to form the wood-based feedstock.

Thus, providing the wood-based feedstock originating from the wood-based raw material may comprise subjecting the wood-based raw material to a mechanical treatment to form a wood-based feedstock. The mechanical treatment may comprise debarking, chipping, dividing, cutting, beating, grinding, crushing, splitting, screening, and/or washing the wood-based raw material. During the mechanical treatment e.g. wood logs can be debarked and/or wood chips of the specified size and structure can be formed. The formed wood chips can also be washed, e.g. with water, in order to remove e.g. sand, grit, and stone material therefrom. Further, the structure of the wood chips may be loosened before the pretreatment step. The wood-based feedstock may contain a certain amount of bark from the wood logs.

Pretreatment of the wood-based feedstock may comprise different pretreatment processes. During the different pretreatment processes the wood-based feedstock as such changes. The aim of the at least one pretreatment processes is to form a fraction comprising solid cellulose particles, i.e. a lignocellulosic material, for further processing.

The pretreatment may comprise subjecting the wood-based feedstock to pre-steaming. The pretreatment may comprise subjecting the wood-based feedstock received from the mechanical treatment to pre-steaming. The pretreatment may comprise, an impregnation treatment and/or a steam explosion and comprise, before subjecting the wood-based feedstock to impregnation treatment and/or to steam explosion, subjecting the wood-based feedstock to pre-steaming. The pre-steaming of the wood-based feedstock may be carried out with steam having a temperature of 100-130° C., at atmospheric pressure. During the pre-steaming the wood-based feedstock is treated with steam of low pressure. The pre-steaming may be also carried out with steam having a temperature of below 100° C., or below 98° C., or below 95° C. The pre-steaming has the added utility of reducing or removing air from inside of the wood-based feedstock. The pre-steaming may take place in at least one pre-steaming reactor.

Further, the pretreatment may comprise subjecting the wood-based feedstock to at least one impregnation treatment with an impregnation liquid. The impregnation treatment may be carried out to the wood-based feedstock received from the mechanical treatment and/or from the pre-steaming. The pretreatment may comprise, before subjecting to the steam explosion, subjecting the wood-based feedstock to at least one impregnation treatment with an impregnation liquid selected from water, at least one acid, at least one alkali, at least one alcohol, or any combination or mixture thereof.

The wood-based feedstock may be transferred from the mechanical treatment and/or from the pre-steaming to the impregnation treatment with a feeder. The feeder may be a screw feeder, such as a plug screw feeder. The feeder may compress the wood-based feedstock during the transfer. When the wood-based feedstock is then entering the impregnation treatment, it may become expanded and absorbs the impregnation liquid.

The impregnation liquid may comprise water, at least one acid, at least one alkali, at least one alcohol, or any combination or mixture thereof. The at least one acid may be selected from a group consisting of inorganic acids, such as sulphuric acid (H₂SO₄), nitric acid, phosphoric acid; organic acids, such as acetic acid, lactic acid, formic acid, carbonic acid; and any combination or mixture thereof. In one embodiment, the impregnation liquid comprises sulphuric acid, e.g. di-lute sulphuric acid. The concentration of the acid may be 0.3-5.0% w/w, 0.5-3.0% w/w, 0.6-2.5% w/w, 0.7-1.9% w/w, or 1.0-1.6% w/w. The impregnation liquid may act as a catalyst in affecting the hydrolysis of the hemicellulose in the wood-based feedstock. In one embodiment, the impregnation is conducted by using only water, i.e. by autohydrolysis. In one embodiment, the wood-based feedstock may be impregnated through alkaline hydrolysis. NaOH and Ca₂(OH)₃ can be mentioned as examples to be used as the alkali in the alkaline hydrolysis.

The impregnation treatment may be conducted in at least one impregnation reactor or vessel. In one embodiment, two or more impregnation reactors are used. The transfer from one impregnation reactor to another impregnation reactor may be carried out with a feeder, such as a screw feeder. The feeder may together with steam even out liquid concentration differences within the wood chips whereby the impregnation liquid may easier penetrate the wood chips.

The impregnation treatment may be carried out by conveying the wood-based feedstock through at least one impregnation reactor, i.e. the wood-based feedstock may be transferred into the impregnation reactor, interspersed inside the impregnation reactor, and transferred out of the impregnation reactor such that the wood-based feedstock is homogenously impregnated with the impregnation liquid. The impregnation treatment may be carried out as a batch process or in a continuous manner.

The residence time of the wood-based feedstock in an impregnation reactor, i.e. the time during which the wood-based feedstock is in contact with the impregnation liquid, may be 5 seconds-5 minutes, or 0.5-3 minutes or about 1 minute. The temperature of the impregnation liquid may be e.g. 20-99° C., or 40-95° C., or 60-93° C. Keeping the temperature of the impregnation liquid below 100° C. has the added utility of hindering or reducing hemicellulose from dissolving.

After the impregnation treatment, the wood-based feedstock may be allowed to stay in e.g. a storage tank or a silo for a predetermined period of time to allow the impregnation liquid absorbed into the wood-based feedstock to stabilize. This predetermined period of time may be 15-60 minutes, or e.g. about 30 minutes. Pretreatment may comprise subjecting the wood-based feedstock to steam explosion. The wood-based feedstock from the mechanical treatment, the pre-steaming step, and/or from the impregnation treatment may be subjected to steam explosion. In one embodiment, pretreatment comprises mechanical treatment of wood-based material to form a wood-based feedstock, the pre-steaming of the wood-based feedstock, impregnation treatment of the pre-steamed wood-based feedstock, and the steam explosion of the impregnated wood-based feedstock. In one embodiment, pretreatment comprises pre-steaming the wood-based feedstock, impregnation treatment of the wood-based feedstock, and steam explosion of the impregnated wood-based feedstock. In one embodiment, pretreatment comprises impregnation treatment of the wood-based feedstock, and steam explosion of the impregnated wood-based feedstock. I.e. the wood-based feedstock having been subjected to the impregnation treatment may thereafter be subjected to the steam explosion. Also, the wood-based feedstock having been subjected to pre-steaming, may then be subjected to the impregnation treatment and thereafter the wood-based feedstock having been subjected to the impregnation treatment may be subjected to steam explosion.

The wood-based feedstock can be stored in e.g. chip bins or silos between the different treatments. Alternatively, the wood-based feedstock may be conveyed from one treatment to the other in a continuous manner.

The pretreatment may comprise subjecting the wood-based feedstock to steam explosion that is carried out by treating the wood-based feedstock with steam having a temperature of 130-240° C. under a pressure of 0.17-3.25 MPaG followed by a sudden, explosive decompression of the wood-based feedstock. The wood-based feedstock may be treated with the steam for 1-20 minutes, or 1-20 minutes, or 2-16 minutes, or 4-13 minutes, or 3-10 minutes, or 3-8 minutes, before the sudden, explosive decompression of the wood-based feedstock.

In this specification, the term “steam explosion” may refer to a process of hemihydrolysis in which the wood-based feedstock is treated in a reactor with steam having a temperature of 130-240° C. under a pressure of 0.17-3.25 MPaG followed by a sudden, explosive decompression of the wood-based feedstock that results in the rupture of the fiber structure of the wood-based feedstock. The output from the steam explosion may be mixed with a suitable liquid, e.g. water, to form a slurry comprising solid cellulose particles. The fraction comprising solid cellulose particles may be separated from the liquid fraction by a suitable separation method, e.g. by a solid-liquid separation.

The steam explosion process may be conducted in a pressurized reactor. The steam explosion may be carried out in the pressurized reactor by treating the wood-based feedstock with steam having a temperature of 130-240° C. under a pressure of 0.17-3.25 MPaG followed by a sudden, explosive decompression of the wood-based feedstock. The wood-based feedstock may be introduced into the pressurized reactor with a compressing conveyor, e.g. a screw feeder. During transportation with the screw feeder, if used, part of the impregnation liquid absorbed by the wood-based feedstock is removed as a pressate while some of it remains in the feedstock. The wood-based feedstock may be introduced into the pressurized reactor along with steam and/or gas. The pressure of the pressurized reactor can be controlled by the addition of steam. The pressurized reactor may operate in a continuous manner or as a batch process. The wood-based feedstock, e.g. the wood-based feedstock that has been subjected to an impregnation treatment, may be introduced into the pressurized reactor at a temperature of 25-140° C. The residence time of the wood-based feedstock in the pressurized reactor may be 0.5-120 minutes. The term “residence time” should in this specification, unless otherwise stated, be understood as the time between the wood-based feedstock being introduced into or entering e.g. the pressurized reactor and the wood-based feedstock being exited or discharged from the same.

As a result of the hemihydrolysis of the wood-based feedstock affected by the steam treatment in the reactor, the hemicellulose present in the wood-based feedstock may become hydrolyzed or degraded into e.g. xylose oligomers and/or monomers. Thus, steam explosion of the wood-based feedstock may result in the formation of an output stream. The output stream from the steam explosion may be subjected to steam separation. The output stream from the steam explosion may be mixed or combined with a liquid. The output stream of the steam explosion may be mixed with a liquid to form a liquid fraction and a fraction comprising solid cellulose particles. The liquid may be pure water or water containing C5 sugars. The water containing C5 sugars may be recycled water from separation and/or washing the fraction comprising solid cellulose particles before enzymatic hydrolysis. The output stream may be mixed with the liquid and the resulting mass may be homogenized mechanically to break up agglomerates.

The liquid fraction may comprise sugars from hydrolyzed hemicellulose as well as soluble lignin and other by-products. In one embodiment, the liquid fraction comprises carbohydrates, such as C5 sugars (C₅H₁₀O₅ or (C₅(H₂O)ₙ). The liquid fraction may comprise carbohydrates, such as monosaccharides (C₆H₁₂O₆ or C₅H₁₀O₅), di-saccharides (C₁₂H₂₂O₁₁), oligosaccharides and/or polysaccharides ((C₆H₁₀O₅)ₙ or (C₅H₈O₄)ₙ). In one embodiment, the liquid fraction comprises soluble C5 carbohydrates (C₅H₁₀O₅ or C₅(H₂O)ₙ) and other carbohydrates. The liquid fraction may comprise also other components.

The fraction comprising solid cellulose particles may, in addition to cellulose, comprise lignin. In one embodiment, the fraction comprising solid cellulose particles comprises carbohydrates, e.g. solid C6 carbohydrates (C₆H₁₂O₆ or C₆(H₂O)ₙ), and lignin. The fraction comprising solid cellulose particles may also comprise other carbohydrates and other components.

The fraction comprising solid cellulose particles is subjected to an enzymatic hydrolysis process which may comprise one or more enzymatic hydrolysis steps. In the enzymatic hydrolysis step, the enzymatic hydrolysis is carried out in at least one enzymatic hydrolysis equipment for forming the hydrolysed material.

In one embodiment, the enzymatic hydrolysis process comprises a pre-hydrolysis step before the first enzymatic hydrolysis step, wherein the fraction comprising solid cellulose particles is subjected to a pre-hydrolysis step for desired time, e.g. 1-2 hours, during which the pH may be kept at a pH value of 3.5-6.5, or 4.0-6.0, or 4.5-5.5The pre-hydrolyzed fraction comprising solid cellulose particles may then as such be directly subjected to the first enzymatic hydrolysis step, i.e. no separation or purification steps are used between the pre-hydrolysis step and the first enzymatic hydrolysis step.

In one embodiment, the first enzymatic hydrolysis step and/or the second enzymatic hydrolysis step are/is carried out at a temperature 30-70° C., or 35-65° C., or 40-60° C., or 45-55° C., or 48-53° C. while keeping the pH of the fraction comprising solid cellulose particles at a pH value of 3.5-6.5, or 4.0-6.0, or 4.5-5.5.

In one embodiment, the first enzymatic hydrolysis step is allowed to continue for 24-72 hours, or 25-40 hours, or 28-31 hours.

In one embodiment, the second enzymatic hydrolysis step is allowed to continue for 24-72 hours, or 32-65 hour, or 35-50 hours, or 38-47 hours.

The enzymes are catalysts for the enzymatic hydrolysis. The enzymatic reaction decreases the pH and by shortening the length of the cellulose fibers it may also decrease the viscosity. Subjecting the lignocellulosic material, such as the fraction comprising solid cellulose particles, to enzymatic hydrolysis may result in cellulose being transformed into glucose monomers with enzymes. Lignin present in the fraction comprising solid cellulose particles may remain essentially in solid form. Cellulose is an insoluble linear polymer of repeating glucose units linked by β-1-4-glucosidic bonds. During the enzymatic hydrolysis, cellulose chains are broken by means of breaking at least one β-1-4-glucosidic bond.

In one embodiment, the hydrolyzed material, from the enzymatic hydrolysis step, e.g. from a first enzymatic hydrolysis step or from a second enzymatic hydrolysis step, is subjected to one or two, or at least three, separation processes or steps in which the solids, such as the solid stream or solid product fraction, and a liquid, such as a solution comprising C6 sugars, are separated. Each of the at least one separation steps, such as decantation, gravitational separation or filtration step, may comprise one, two or more decanter, gravitational separator, filtration device or their combination, such as parallel decanters, gravitational separators or filtration devices. In one embodiment, the decantation step or each of the decantation steps comprises one or at least two or more parallel decanters. In one embodiment, the gravitational separation step or each of the gravitational separation steps comprises one or at least two or more parallel gravitational separators. In one embodiment, the filtration step or each of the filtration steps comprises one or at least two or more parallel filtration devices. The separation process may comprise re-slurring the solid stream and thereafter subjecting the formed slurry to a next separation step.

In the method, the steps i) and/or ii) may be carried out once, twice or several times. The separation of the solid stream can be carried out at one or more steps. The flocculant additive can be added in one or more points or steps of the process, for example before the decantation or gravitational separation step, between two decantation or gravitational separation steps or after the decantation or gravitational separation step.

The flocculant additive may be added in one, two or more points of the process. In one embodiment, the flocculant additive is added at least before the first decantation or gravitational separation step. In one embodiment, the process comprises at least two decantation or gravitational separation steps and the flocculant additive is added at least before the first decantation or gravitational separation step and/or at least before the second decantation or gravitational separation step. In one embodiment, the flocculant additive is added at least after the first decantation or gravitational separation step. In one embodiment, the flocculant additive is added at least after the second or any later decantation or gravitational separation step. In one embodiment, the flocculant additive is added between two decantation or gravitational separation steps. In one embodiment, the flocculant additive is added at least after the enzymatic hydrolysis or the enzymatic hydrolysis step. In one embodiment, the process comprises at least two enzymatic hydrolysis steps and the flocculant additive is added after the first enzymatic hydrolysis step and/or after the second enzymatic hydrolysis step.

In this context, any suitable flocculant additive can be used. In one embodiment, the flocculant additive is a cationic polymer flocculant or another suitable flocculant. The flocculant additive usually in a form of fine particulate powder. Before adding it to the hydrolyzed material or a stream separated from the hydrolyzed material the flocculant additive is mixed with water.

In one embodiment, the mixture is treated by means of the decantation in the decantation step after the enzymatic hydrolysis. In one embodiment, at least the mixture is treated by means of the gravitational separation in the gravitational separation step after the enzymatic hydrolysis. In one embodiment, the mixture is treated by means of the decantation and the gravitational separation after the enzymatic hydrolysis. The decantation and/or the gravitational separation may be carried out in one or more separation steps. Each of the decantation or gravitational separation steps may comprise one or more decanters or gravitational separators, e.g. parallel decanters or gravitational separators. In one embodiment, the hydrolyzed material or the stream separated from the hydrolyzed material is treated in one or more decanter or gravitational separator, in which the liquid stream is separated from the solid stream.

In this context, the solid stream means any solid stream, solid fraction or solid-based stream separated in any decantation and/or gravitational separation step. In this context, the liquid stream means any liquid stream or liquid fraction separated in any decantation and/or gravitational separation step or in any filtration step, e.g. by a filter press. In this context, the stream separated from the hydrolyzed material may mean any separated stream or fraction of the hydrolyzed material, for example a solid fraction or the solid stream, which may be re-slurred.

In one embodiment, the method comprises at least two decantation or gravitational separation steps, and the mixture is subjected to a first decantation or gravitational separation step, the solid stream, i.e. a stream separated from the hydrolyzed material, from the first decantation or gravitational separation step is re-slurried and the re-slurried solid stream is subjected to the second decantation or gravitational separation step.

In one embodiment, the solid stream from the decantation or gravitational separation step, e.g. after the first separation step, is re-slurried and supplied to the next enzymatic hydrolysis step. In one embodiment, the method comprises at least two enzymatic hydrolysis steps, and the hydrolyzed material is treated after the first enzymatic hydrolysis step by adding the flocculant additive and/or separating the solid stream in the decantation or gravitational separation step, the solid stream is re-slurried and subjected to the second enzymatic hydrolysis to form a second hydrolyzed material. The second hydrolyzed material may be treated after the second enzymatic hydrolysis step by adding the flocculant additive to the second hydrolyzed material and/or by separating a second solid stream and a second liquid stream from the second hydrolyzed material in a second decantation or gravitational separation step. After second decantation or gravitational separation step, the second solid stream may be re-slurried and subjected to the next decantation or gravitational separation step for separating a next solid stream. In one embodiment, the method comprises a first enzymatic hydrolysis step, after which the hydrolyzed material is treated according to the steps i) and ii) to form a solid stream and the solid stream is re-slurried, and the method comprises a second enzymatic hydrolysis step, to which the re-slurried solid stream is subjected to form a second hydrolyzed material and after which the second hydrolyzed material is treated by adding the flocculant additive to the second hydrolyzed material and by separating a second solid stream and a second liquid stream from the second hydrolyzed material in a second decantation or gravitational separation step. In one embodiment, the second solid stream is re-slurried and subjected to the third decantation or gravitational separation step for separating a third solid stream. In one embodiment, the enzymatic hydrolysis step comprises one or more enzymatic hydrolysis equipments. In one embodiment, the method comprises at least two enzymatic hydrolysis steps, and the hydrolyzed material is treated after the both enzymatic hydrolysis steps by adding the flocculant additive and separating the solid stream in at least one or more decantation or gravitational separation steps.

The separated solid stream from the decantation or gravitational separation step may be re-slurried with a dilution liquid before a next step, e.g. next decantation, gravitational separation or solid-liquid separation or next enzymatic hydrolysis step. In a re-slurrying, the dilution liquid is added to the solid stream and a mixture is mixed for forming a slurry. In one embodiment, the method comprises the re-slurrying in which the solid stream after the decantation or gravitational separation step is mixed with the dilution liquid to form a re-slurried solid stream, such as the slurry. The solid stream does not dissolve in the dilution liquid. In this context, by the expression “dilution liquid” should be understood, unless otherwise stated, as referring to water, liquid stream separated by decantation, gravitational separation or filtration step or any mixture or combination thereof. In this context, by the expression “recycled liquid stream” should be understood, unless otherwise stated, as referring to liquid stream separated by decantation, gravitational separation or filtration step or any mixture or combination thereof, that is fed back to any point of the process. In one embodiment, the dilution liquid is water. In one embodiment, the dilution liquid is a recycled liquid stream. In one embodiment, the process arrangement comprises at least one re-slurring tank for re-slurring the solid stream, for example before the next decanter or gravitational separator, or before the next enzymatic hydrolysis equipment, or before the separation in the filtration device. In one embodiment, the dilution liquid is added to achieve the TS of 5-15% in the re-slurried solid stream.

In one embodiment, the dilution liquid is added to the solid stream of the decantation or gravitational separation in the re-slurring step. In one embodiment, at least a part of the liquid stream, such as the recycled liquid stream, from the decantation or gravitational separation step or from the filtration step is recirculated to the enzymatic hydrolysis, such as to the first enzymatic hydrolysis or pre-hydrolysis, and/or to the re-slurring step. In one embodiment, the process arrangement further comprises at least one recirculating device, e.g. a recirculation line, for recirculating the recycled liquid stream from the decanter or gravitational separator or from the filtration device to the enzymatic hydrolysis, e.g. to the first enzymatic hydrolysis or pre-hydrolysis, and/or to the re-slurring tank. The recycled liquid stream may be the liquid stream from the decanter or gravitational separator or from the filtration device, or a part of the said liquid stream.

The solids may be separated and recovered by means of at least one device. This device may be the decanter or gravitational separator, or the filtration device. In one embodiment, the process arrangement comprises at least one filtration device for separating a solid product fraction from the re-slurried solid stream after the last decanter or gravitational separator. The method and process arrangement may comprise one or more filtration steps, comprising at least one filtration device, after the last decantation or gravitational separation step. Each of the filtration steps may comprises one or more filtration devices, e.g. parallel devices. The filtration device may be any filtration device, separating device, separator, filter press, pressure filter, vacuum filtration device, or another separation device, where the solids, such as the solid product fraction, can be separated from liquid. In one embodiment, the method comprises separating the solid product fraction from the re-slurried solid stream by means of the filtration device, e.g. filter press, in the filtration step after the decantation or gravitational separation step. In one embodiment, the solid stream of the decantation or gravitational separation step is re-slurried and a solid additive is added to the re-slurried solid stream. In one embodiment, the solid stream of the decantation or gravitational separation step is re-slurried, the solid additive is added to the re-slurried solid stream and the solid product fraction is separated from the re-slurried solid stream by means of the filtration device in the filtration step. In one embodiment, the process arrangement comprises a second feeder for feeding a solid additive to the solid stream of the decanter or gravitational separator before the filtration device. In one embodiment, the solid additive is added in connection with the re-slurring step. In one embodiment the solid product fraction comprise lignin. In one embodiment, the solid product fraction is a lignin product.

The solid additive may be any solid additive, solid filtering aid or another suitable additive which comprises fine particles. In one embodiment, the solid additive is a bark, wood powder from a wood chips screening, biomaterial, biomass particles, infusioral earth particles, or material which filters easily and/or facilitates filtration. The solid additive may be in the form of solid or slurry.

In one embodiment, a residual solid stream is separated, e.g. by means of disc stack separator, from a liquid stream after the decantation or gravitational separation step. In one embodiment, the process arrangement comprises at least one separator for separating the residual solid stream from the liquid stream after the decanter or gravitational separator. In one embodiment, the flocculant additive is added to the liquid stream of the decantation or gravitational separation step before separating the residual solid stream from the liquid stream. In one embodiment, the feeder is arranged to feed the flocculant additive to the liquid stream of the decanter or gravitational separator before the separator in which the residual solid stream is separated from the liquid stream.

In one embodiment, the flocculant additive is added before the decantation, gravitational separation or filtration step. In one embodiment, the flocculant additive is added to the hydrolyzed material before the decantation or gravitational separation step. In one embodiment, the flocculant additive is added to the re-slurried solid stream of the decantation or gravitational separation, e.g. before the next decantation or gravitational separation step. In one embodiment, the flocculant additive is added in connection with the reslurring step. In one embodiment, the feeder is arranged to feed the flocculant additive to the solid stream of the decanter or gravitational separator in connection with the re-slurring tank.

The flocculant additive may be added to a desired point of the process. In one embodiment the flocculant additive is added to the hydrolyzed material or the re-slurried solid stream of the decantation or gravitational separation, e.g. before the next decantation or gravitational separation step. By means of the flocculant additive, e.g. cationic polymer flocculant, the small solid particles in the liquid are drawn to each other and form bigger flocs that can be easily removed.

In one embodiment, the flocculant additive is fed to a feed of a pump located before the decantation or gravitational separation step, to a feeding line between the pump and the decantation or gravitational separation step, to the decantation or gravitational separation step, or just before a mixer or an in-line mixer located before the decanter or gravitational separator. In one embodiment, the feeder is arranged to feed the flocculant additive to a feed of a pump located before the decanter or gravitational separator, to a feeding line between the pump and the decanter or gravitational separator, to the decanter or gravitational separator, or just before a mixer or an in-line mixer located before the decanter or gravitational separator. The inventors surprisingly found out that when the flocculant additive is added, the separation of the solid streams can be improved in the decanters and gravitational separators, and for example in disc stack separators. Further, when the flocculant additive is added, enzymes are retained in the solid material, and thus, there is no enzyme recycling in the process. Further, the inventors surprisingly found out that using centrifugal or gravitational type separators, e.g. decanter centrifuge, alone without the flocculant additive in the process described in this application, poor separation efficiency is achieved. The solid particles in the hydrolyzed material and in the streams separated from the hydrolyzed material are small and light weight, and the flocculant is needed to achieve an adequate separation. In one embodiment, the flocculant additive is added to the hydrolyzed material after the enzymatic hydrolysis. In one embodiment, the flocculant additive is added to the hydrolyzed material after the enzymatic hydrolysis, and after that the hydrolyzed material is treated by means of the decantation in at least one decanter by separating a solid stream, e.g. first solid stream, from a liquid stream in the decantation step. In one embodiment, the flocculant additive is added to the hydrolyzed material after the enzymatic hydrolysis, and after that the hydrolyzed material is treated by means of the gravitational separation in at least one gravitational separator by separating a solid stream, e.g. first solid stream, from a liquid stream in the gravitational separation step.

In one embodiment, the hydrolyzed material is treated by means of the decantation or gravitational separation by separating a solid stream, e.g. first solid stream, from a liquid stream. The flocculant additive may be added to the hydrolyzed material after the enzymatic hydrolysis step. The solid stream, e.g. the first solid stream, after the first decantation or gravitational separation step is re-slurried, and after that the re-slurried solid stream is treated by means of the second decantation or gravitational separation in at least one decanter or gravitational separator by separating a second solid stream from a second liquid stream in the second decantation or gravitational separation step. The flocculant additive may be added to the re-slurried solid stream before the second decantation or gravitational separation step. The re-slurring and the treatment by the second decantation or gravitational separation can be repeated once or more times. In one embodiment, the flocculant additive can be added before the desired decantation and/or gravitational separation steps. In one embodiment the solids, preferably with high purity and high solids content, are separated from the last solid stream of the last decantation or gravitational separation step by means of the filtration device, e.g. filter press or other suitable solid-liquid separator, in a filtration step. The last solid stream may be re-slurried before the separation in the filtration device.

In one embodiment, the process comprises at least two enzymatic hydrolysis steps, and the hydrolyzed material of the first enzymatic hydrolysis step is treated by means of the decantation or gravitational separation by separating a solid stream, e.g. first solid stream, from a liquid stream in the decantation or gravitational separation step, and the first solid stream after the first decantation or gravitational separation step is re-slurried, and after that the re-slurried solid stream is subjected to the second enzymatic hydrolysis step. The flocculant additive may be added to the hydrolyzed material after the first enzymatic hydrolysis step. In one embodiment, after the second enzymatic hydrolysis step, the flocculant additive is added to the second hydrolyzed material of the second enzymatic hydrolysis step, and after that the second hydrolyzed material is treated by means of the decantation in at least one decanter or the gravitational separation in at least one gravitational separator in a second decantation or gravitational separation step by separating a second solid stream from a liquid stream. In one embodiment, the second solid stream is re-slurried, and after that the re-slurried second solid stream is treated by means of the decantation in at least one decanter or the gravitational separation in at least one gravitational separator in a third decantation or gravitational separation step by separating a third solid stream from a liquid stream. The re-slurring and the treatment by the decantation or gravitational separation can be repeated once or more times. In one embodiment, the flocculant additive can be added before the desired the decantation or gravitational separation steps. In one embodiment, the solids preferably with high purity and high solids content are separated from the last solid stream of the last decantation or gravitational separation step, e.g. from the third solid stream, by means of at least one solid-liquid separator, e.g. filter press or other suitable solid-liquid separator, in a filtration step. The last solid stream may be re-slurried before the separation in the solid-liquid separator.

In one embodiment, a solid stream of the hydrolyzed material is separated, e.g. by a decantation or gravitational separation step, from a liquid stream of the hydrolyzed material after the enzymatic hydrolysis step, the solid stream is re-slurried and the additive is added to the said re-slurried solid stream, and after that the re-slurried solid stream is treated by means of the next decantation in at least one decanter or the gravitational separation in at least one gravitational separator by separating a second solid stream from a liquid stream. The re-slurring and the treatment by the decantation or gravitational separation can be repeated once or more times. The addition of the additive can be also repeated between the re-slurring and the separation.

In one embodiment, a solid stream and a liquid stream of the hydrolyzed material are separated by a decantation or gravitational separation step after the enzymatic hydrolysis step, and the additive is added to the separated liquid stream, and after that a residual solid stream is separated from a liquid stream, such as from a clear liquid stream, by means of at least one solid-liquid separator, e.g. disc stack separator or other suitable solid-liquid separator.

In one embodiment, the solid additive is added to the solid stream in connection with the re-slurrying. In one embodiment, the solid additive is added during a mixing of the solid stream and dilution liquid, or in a mixer or an in-line mixer, or just before the mixing or a mixer or in-line mixer.

In one embodiment, the solid additive is added to the solid stream, e.g. the solid fraction from the enzymatic hydrolysis step or the solid stream from the latest decantation or gravitational separation step, before the filtration step, e.g. separation by a filter press or another separation device, in which the solids, e.g. solid product fraction, are separated. The solid additive is added in connection with the re-slurrying or during the re-slurrying.

In one embodiment, the solid additive is added to the solid stream of the last decantation or gravitational separation step during the re-slurrying before the filtration step, and the solids with high solids content are separated in at least one filtration device, e.g. filter press or other suitable solid-liquid separator, in the filtration step.

In one embodiment, temperature is arranged to 55-75° C., in one embodiment to 60-70° C. and in one embodiment to 65-70° C., during the re-slurring, e.g. in the re-slurrying step, in which the solid additive is added. A material of the re-slurrying and/or components fed to the re-slurrying may be heated to achieve the desired temperature in the re-slurrying. During the re-slurring at the said temperatures a net of material components formed by the flocculant, e.g. cationic polymer flocculant, can be broken down in the solid material of the solid stream before the solid-liquid separation. Further, the enzymes which are in the solid stream can be inactivated at high temperature during the re-slurring.

The inventors surprisingly found out that when the temperature is 55-75° C. during the re-slurring before the solid-liquid separation, e.g. in the filter press, filtration can be improved in the said solid-liquid separation. Further, the inventors surprisingly found out that when the solid additive is added, the solid-liquid separation can be improved in the filtration device.

In one embodiment, the liquid stream received from the decantation or gravitational separation step, e.g. from the latest decantation or gravitational separation step, may be recirculated as a dilution liquid to the pre-hydrolysis step, to the enzymatic hydrolysis step and/or to desired re-slurrying or re-slurrying step. In one embodiment, the liquid stream received from the filtration step may be recirculated as a dilution liquid to the pre-hydrolysis step, to the enzymatic hydrolysis step and/or to desired re-slurrying or re-slurrying step.

The solid product including the solids can be formed by the method and process arrangement. The solid product contains at least lignin. In one embodiment, the solid product is lignin, and it comprises 0.5-6 weight-% of soluble components. In one embodiment, the solid product comprises acid-insoluble lignin in an amount of 80-90 weight-% based on the total dry matter content of the lignin composition, wherein the average molecular weight of the lignin is 5000-15000 Da. In one embodiment, the solid product comprises carbohydrates in an amount of 1.5-15 weight-% based on the total dry matter content of the lignin composition.

Further, the liquid stream comprising C₆ carbohydrates may be recovered.

The solid product can be used for the production of a composite, a filler material, an adhesive, a paint, or a resin, or other suitable product.

The cationic polymer flocculant can be used as an additive for improving a separation of solids from a hydrolyzed material formed in the enzymatic hydrolysis of lignocellulosic material and for dewatering of the solids of the hydrolyzed material.

The solid additive, such as bark, wood powder from a wood chips screening, biomaterial, biomass particles, infusioral earth particles or another suitable solid material, can be used as an additive for improving a separation of solids from a hydrolyzed material formed in the enzymatic hydrolysis of lignocellulosic material, for facilitating the separation, such as filtration, of the solids after the decantation or gravitational separation, or for facilitating a recovery of the solids.

The method and process arrangement as disclosed in the current specification has the added utility of separating the fractions after the enzymatic hydrolysis.

Thanks to the method and process arrangement the continuous process can be provided, and simultaneously contamination can be decreased in the process, and thus the process can be improved. Further, the separation of the solids and liquids can be improved. The solids can be recovered more efficiently. The solid product with high purity and high solids content can be produced. The method and process arrangement offer a possibility to separate the solid and liquid streams easily and effectively. The method and process arrangement are easy and simple to realize in connection with production processes.

### EXAMPLES

Reference will now be made in detail to the embodiments of the present disclosure, some examples of which are illustrated in the accompanying drawings.

The description below discloses some embodiments in such a detail that a person skilled in the art is able to utilize the method and process arrangement based on the disclosure. Not all steps of the embodiments are discussed in detail, as many of the steps will be obvious for the person skilled in the art based on this disclosure.

For reasons of simplicity, item numbers will be maintained in the following exemplary embodiments in the case of repeating components.

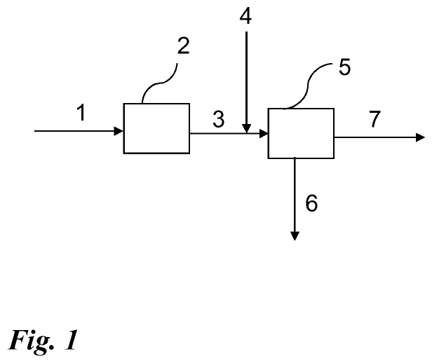

The enclosed FIGS. 1-5 illustrate embodiments of the method and process arrangement for improving a solid-liquid separation of solids from a hydrolyzed material in some detail.

The lignocellulosic material (1), such as the fraction comprising solid cellulose particles, may be provided e.g. from a wood-based feedstock originating from wood-based raw material and comprising wood chips, that is subjected to at least one pretreatment to form a liquid fraction and a fraction comprising solid cellulose particles. The liquid fraction and the fraction comprising solid cellulose particles may be then separated e.g. by a solid-liquid separation process. The provided fraction comprising solid cellulose particles is then subjected to a first enzymatic hydrolysis step for 8-72 hours to form a hydrolyzed material (3). The solid and liquid fractions may be then separated. The separated solid fraction, such as a solid stream, may be then re-slurried, and the re-slurried solid fraction is then subjected to a second enzymatic hydrolysis step for 8-72 hours to form a second hydrolyzed material (15). The hydrolyzed material is a product of the enzymatic hydrolysis which may comprise the first enzymatic hydrolysis step (2), or the first (2) and second (14) enzymatic hydrolysis steps.

The method and process arrangement of FIG. 1 comprises adding a flocculant additive (4), such as cationic polymer flocculant, to the hydrolyzed material (3) after the enzymatic hydrolysis step (2) and after that treating the hydrolyzed material by means of a decantation (5) or the gravitational separation in at least one decanter or gravitational separator by separating a solid stream (6), e.g. first solid stream, from a liquid stream (7).

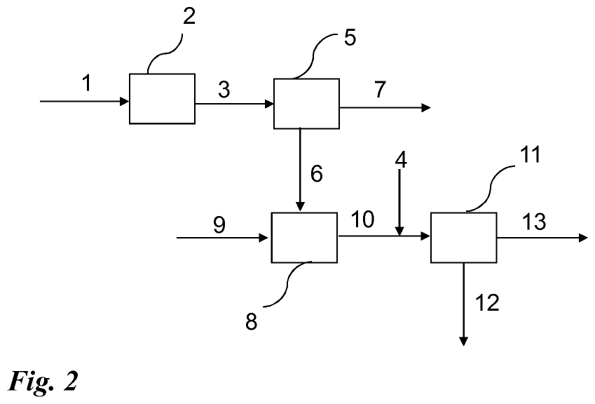

The method and process arrangement of FIG. 2 comprises treating the hydrolyzed material (3) of the enzymatic hydrolysis step (2) by means of the decantation (5) or the gravitational separation in at least one decanter or gravitational separator by separating a first solid stream (6) from a liquid stream (7), and re-slurring (8) the first solid stream in a re-slurring step (8). A dilution liquid (9), e.g. water or recycled liquid stream, is fed to the re-slurring step (8). Further, the method and process arrangement comprises adding the flocculant additive (4), such as cationic polymer flocculant, to a re-slurried solid stream (10) and treating the re-slurried solid stream (10) by means of the second decantation (11) or gravitational separation in at least one second decanter or gravitational separator for separating a second solid stream (12) from a second liquid stream (13). The re-slurring, the adding of the flocculant additive and/or the separation by the second decantation or gravitational separation can be repeated. The solids, preferably with high purity and high solids content, may be separated from the last solid stream of the last decantation or gravitational separation by means of at least one solid-liquid separator, e.g. filter press or other suitable solid-liquid separator. The last solid stream may be re-slurried before the separation in the solid-liquid separator.

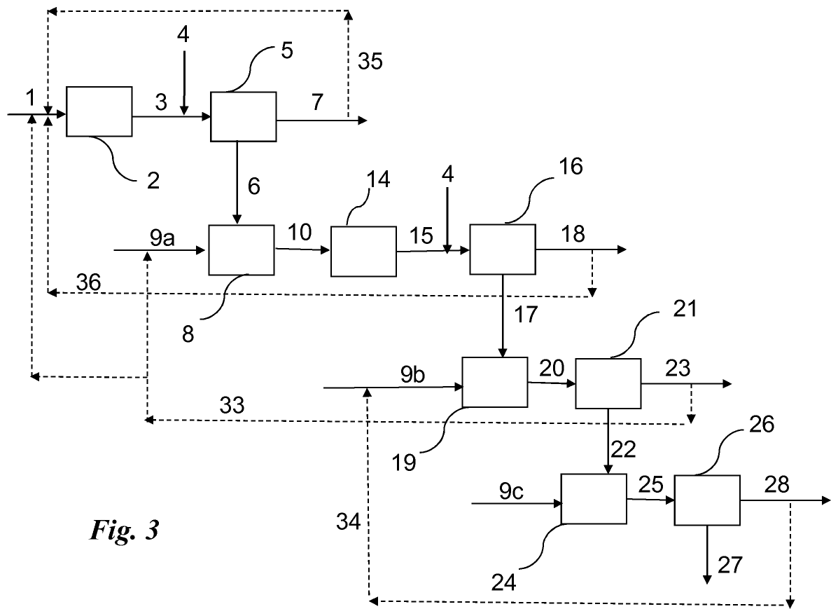

The method and process arrangement of FIG. 3 comprises two enzymatic hydrolysis steps. It also comprises adding a flocculant additive (4), such as cationic polymer flocculant, to the hydrolyzed material (3) after the first enzymatic hydrolysis step (2), and treating the hydrolyzed material by means of the decantation (5) or gravitational separation in the decantation or gravitational separation step, comprising at least one decanter or gravitational separator, e.g. three parallel devices, by separating a first solid stream (6) from a first liquid stream (7), and after that re-slurring (8) the first solid stream, and subjecting the re-slurried first solid stream (10) to the second enzymatic hydrolysis step (14). A first dilution liquid (9a), e.g. water and/or recycled liquid stream (33), is fed to the first re-slurring step (8) to form the re-slurried first solid stream (10). After the second enzymatic hydrolysis step, the flocculant additive (4) is added to the second hydrolyzed material (15), and the second hydrolyzed material is treated by means of the second decantation (16) or the gravitational separation by separating a second solid stream (17) from a second liquid stream (18) in the second decantation or gravitational separation step, comprising at least one decanter or gravitational separator, e.g. two parallel devices. The second solid stream is re-slurried in a second re-slurring step (19) with a second dilution liquid (9b), e.g. water and/or recycled liquid stream (34), to form a re-slurried second solid stream (20), and after that the re-slurried second solid stream (20) is treated by means of the third decantation (21) or the gravitational separation by separating a third solid stream (22) from a third liquid stream (23) in the third decantation or gravitational separation step, comprising at least one decanter or gravitational separator, e.g. two parallel devices. The third solid stream (22) is re-slurried in a third re-slurring step (24) with a third dilution liquid (9c) which is water to form a re-slurried third solid stream (25), and the re-slurried third solid stream (25) is subjected to a filtration step (26) comprising at least one filtration device, e.g. filter press or other suitable solid-liquid separator, for separating the solid product fraction (27), e.g. the solids, with high purity and high solids content from a fourth liquid stream (28). A part of the first liquid stream (7), comprising mainly C6 carbohydrates, may be recirculated as a dilution liquid (35) to the first enzymatic hydrolysis step or a pre-hydrolysis. The second liquid stream (18), comprising also mainly C6 carbohydrates, or a part of this may be recirculated as a dilution liquid (36) to the first enzymatic hydrolysis step or a pre-hydrolysis. The third liquid stream (23) or a part of this may be recirculated as a dilution liquid (33) to the first re-slurrying step (8) and/or to the first enzymatic hydrolysis step or a pre-hydrolysis. The fourth liquid stream (28) or a part of this may be recirculated as a dilution liquid (34) to the previous, second re-slurrying step (19).

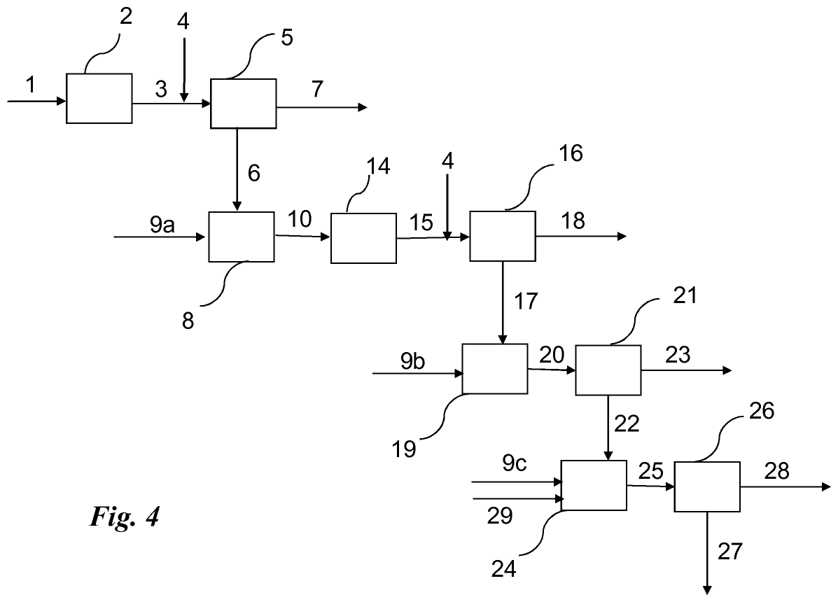

The method and process arrangement of FIG. 4 comprises adding a flocculant additive (4), such as cationic polymer flocculant, to the hydrolyzed material (3) after the first enzymatic hydrolysis step (2), and treating the hydrolyzed material by means of the decantation (5) or gravitational separation by separating a first solid stream (6) from a first liquid stream (7), and after that re-slurring (8) the first solid stream, and subjecting the re-slurried first solid stream (10) to the second enzymatic hydrolysis step (14). A first dilution liquid (9a), e.g. water and/or recycled liquid stream, is fed to the first re-slurring step (8) to form the re-slurried first solid stream (10). After the second enzymatic hydrolysis step, the flocculant additive (4) is added to the second hydrolyzed material (15), and the second hydrolyzed material is treated by means of the second decantation (16) or the gravitational separation by separating a second solid stream (17) from a second liquid stream (18). The second solid stream is re-slurried in a second re-slurrying step (19) with a second dilution liquid (9b), e.g. water or recycled liquid stream, to form a re-slurried second solid stream (20), and after that the re-slurried second solid stream (20) is treated by means of the third decantation (21) or the gravitational separation by separating a third solid stream (22) from a third liquid stream (23). The third solid stream (22) is re-slurried in a third re-slurrying step (24) with a third dilution liquid (9c) which is water to form a re-slurried third solid stream (25), and a solid additive (29), e.g. bark, is added to the third solid stream (22) during the third re-slurring step, and the re-slurried third solid stream (25) is subjected to a solid-liquid separator (26), e.g. filter press or other suitable solid-liquid separator, for separating the solid product fraction (27), such as the solids, with high purity and high solids content from a fourth liquid stream (28). The first, second, third and fourth liquid streams may be recirculated to the desired re-slurring step or the enzymatic hydrolysis step or pre-hydrolysis, as in FIG. 3. Temperature can be arranged to 55-75° C. during the re-slurring in the third re-slurrying step (24) in which the solid additive (29) is added.

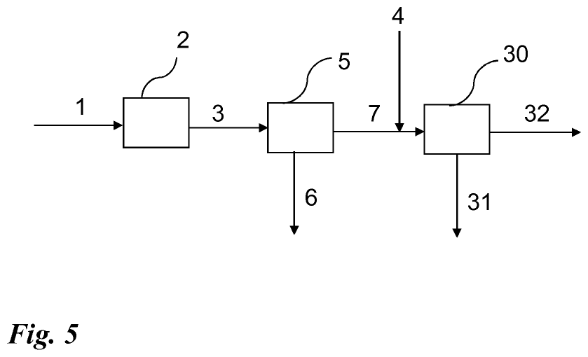

The method and process arrangement of FIG. 5 comprises treating the hydrolyzed material (3) of the enzymatic hydrolysis step (2) by means of the decantation (5) or the gravitational separation in a decanter or a gravitational separator by separating a solid stream (6) from a liquid stream (7), and adding the flocculant additive (4), such as cationic polymer flocculant, to the liquid stream (7), and after that separating a residual solid stream (31) from a clear liquid stream (32) in a disc stack separator (30).

### Example 1

In this example, the solid-liquid separation was carried out by a centrifugal decanter for separating solids from a hydrolyzed material.

A feed, which was the hydrolyzed material from an enzymatic hydrolysis, was subjected to the decanter in which the solids (cake) and a liquid fraction were separated. The material was pretreated with a diluted acid treatment and steam explosion and washed for separating C5-carbohydrates from the material before the enzymatic hydrolysis. The enzymatic hydrolysis comprised two stages, a first enzymatic hydrolysis and a second enzymatic hydrolysis.

In trials 27-32, the feed was the hydrolyzed material from the first enzymatic hydrolysis stage, which was subjected to a first decanter as feed material. After the trials 27-32, the separated cake materials of the trial runs 27-32 were combined, re-slurried and subjected to the second enzymatic hydrolysis. In trials 33-36, the feed to a second decanter was the hydrolyzed material from the second enzymatic hydrolysis step. After the trials 33-36, the separated cake materials of the trial runs 33-36 were combined, re-slurried with water and fed to a third decanter to be used as feed material for the trial runs 37-39.

The flocculant additive (flocculant), having concentration 0.2%, was added to the feed before the solid-liquid separation in trials 27-38. In trial 39, the flocculant additive was not added. The cationic polymer flocculant (polyacrylamide) was used as the flocculant. The flocculant additive was formed by suspending the dry flocculant with water.

The process conditions and the results are presented in tables 1-3.

The TS (total solids at 105° C.) and SS (suspended solids) of the feed material to the decanters in the trials were as follows:

| Trial numbers | TS (weight-%) | SS (weight-%) |
|---|---|---|
| 27-32 | 13.4 | 8.2 |
| 33-36 | 12.9 | 7.4 |
| 37-39 | 10.3 | 8.0 |

TABLE 1

| | Feed | Feed | Floc | | | Solid in |
|---|---|---|---|---|---|---|
| Trial | flow | density | dosing | Cake | Cake | liquid |
| point | m³/h | kg/l | kg/t TS | TS | SS | fraction mg/l |
| 27 | 4 | 1.03 | 1.5 | 29.5 | 25.8 | 3 |
| 28 | 5 | 1.03 | 1.5 | 27.0 | 23.2 | 69 |
| 29 | 6 | 1.03 | 1.5 | 28.9 | 25.2 | 59 |
| 30 | 7 | 1.03 | 1.5 | 29.8 | 26.1 | 67 |
| 31 | 8 | 1.03 | 1.5 | 31.6 | 28.0 | 67 |
| 32 | 8 | 1.03 | 1.0 | 29.5 | 25.8 | 880 |

TABLE 2

| | Feed | Feed | Floc | | | Solid in |
|---|---|---|---|---|---|---|
| Trial | flow | density | dosing | Cake | Cake | liquid |
| point | m³/h | kg/l | kg/t TS | TS | SS | fraction mg/l |
| 33 | 6 | 1.02 | 1.5 | 30.3 | 26.5 | 64 |
| 34 | 7 | 1.02 | 1.5 | 28.6 | 24.7 | 37 |
| 35 | 8 | 1.02 | 1.5 | 30.5 | 26.7 | 83 |
| 36 | 8.5 | 1.02 | 1.5 | 30.6 | 26.8 | 64 |

TABLE 3

| | Feed | Feed | Floc | | | Solid in |
|---|---|---|---|---|---|---|
| Trial | flow | density | dosing | Cake | Cake | liquid |
| point | m³/h | kg/l | kg/t TS | TS | SS | fraction mg/l |
| 37 | 6 | 1.02 | 1.5 | 29.0 | 27.4 | 41 |
| 38 | 8 | 1.02 | 1.5 | 28.6 | 27.0 | 29 |
| 39 | 8.5 | 1.02 | 0 | 29.9 | 28.3 | 2264 |

TS = Total solids at 105° C.
SS = Suspended solids
Floc dosing = Dosing of flocculants, kg/t TS (dry flocculant/solid material)

It was observed from the trials that the solid-liquid separation in the decanter can be improved when the flocculant additive was added before the separation. Both losses in carbohydrate (liquid fraction) yield and solids (lignin) yield could be decreased when the flocculant was used. If the flocculant additive was not added, as in trial 39, the solids of the liquid fraction increased strongly. Further, it was observed that the liquid fractions with the solid content of below 100 mg/l can be produced when the flocculant was added to the feed. Then the solids can be recovered better when the flocculant is used in the process. Further, it was observed that the liquid fraction contained more solids when the dosing of the flocculant decreases (trial point 32).

Further, in the following tests 1, 2, 3 and 4, the combined cakes from trial runs 37-39 of table 3 were re-slurried to form a slurry, and the slurry was subjected to a pressure filtration. Total solids (TS) of the slurry was 13.2% and suspended solids (SS) of the slurry was 11.4%. Temperature of the slurry was 69.9° C. and pH of the slurry was 5.0 before the pressure filtration. In the pressure filtration the slurry was filtrated such that the slurry was pumped and pressed, and a filtration cake was formed.

In test 1 (chamber 40 mm), time of the pumping was 4.5 min and pressure at the end of the pumping was 5.0 bar. Time of the pressing was 5.0 min and pressure at the end of the pressing was 12.0 bar. The filtration cake after the pressing was 3.6 kg, thickness of the filtration cake was 27-30 mm, and dry matter of the filtration cake was 54.70%. An amount of a filtrate was 14.7 kg.

In test 2 (chamber 40 mm), time of the pumping was 3.0 min and pressure at the end of the pumping was 5.0 bar. Time of the pressing was 3.0 min and pressure at the end of the pressing was 8.0 bar. The filtration cake after the pressing was 3.75 kg, thickness of the filtration cake was 27-30 mm, and dry matter of the filtration cake was 50.70%. An amount of a filtrate was 13.7 kg.

In test 3 (chamber 50 mm), time of the pumping was 3.0 min and pressure at the end of the pumping was 5.0 bar. Time of the pressing was 3.0 min and pressure at the end of the pressing was 8.0 bar. The filtration cake after the pressing was 4.75 kg, thickness of the filtration cake was 36 mm, and dry matter of the filtration cake was 49.90%. An amount of a filtrate was 17.2 kg.

In test 4 (chamber 50 mm), time of the pumping was 3.0 min and pressure at the end of the pumping was 5.0 bar. Time of the pressing was 2.0 min and pressure at the end of the pressing was 8.0 bar. The filtration cake after the pressing was 4.8 kg, thickness of the filtration cake was 33-38 mm, and dry matter of the filtration cake was 49.20%. An amount of a filtrate was 16.8 kg.

It was observed from the pressure filtration tests that the flocculants, which were added in previous decantation steps, did not disturb the filtration, and the filtration could be performed effectively.

It is obvious to a person skilled in the art that with the advancement of technology, the basic idea may be implemented in various ways. The embodiments are thus not limited to the examples described above; instead they may vary within the scope of the claims.

The embodiments described hereinbefore may be used in any combination with each other. Several of the embodiments may be combined together to form a further embodiment. A method or process arrangement disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items. The term “comprising” is used in this specification to mean including the feature(s) or act(s) followed thereafter, without excluding the presence of one or more additional features or acts.

1. A method for improving a solid-liquid separation of solids from a hydrolyzed material formed in an enzymatic hydrolysis of lignocellulosic material, wherein the method comprises:
i) adding a flocculant additive to the hydrolyzed material or to a stream separated from the hydrolyzed material to form a mixture; and
ii) separating a solid stream and a liquid stream from the mixture by means of a decantation or gravitational separation.

2. The method according to claim 1, wherein the method further comprises a re-slurrying in which the solid stream after the decantation or gravitational separation step is mixed with a dilution liquid to form a re-slurried solid stream.

3. The method according to claim 1, wherein the method comprises at least two decantation or gravitational separation steps, and the mixture is subjected to a first decantation or gravitational separation step, the solid stream from the first decantation or gravitational separation step is re-slurried and the re-slurried solid stream is subjected to the second decantation or gravitational separation step.

4. The method according to claim 1, wherein the method further comprises:
a first enzymatic hydrolysis step, after which the hydrolyzed material is treated according to the steps i) and ii) to form a solid stream and the solid stream is re-slurried; and
a second enzymatic hydrolysis step, to which the re-slurried solid stream is subjected to form a second hydrolyzed material and after which the second hydrolyzed material is treated by adding the flocculant additive to the second hydrolyzed material and by separating a second solid stream and a second liquid stream from the second hydrolyzed material in a second decantation or gravitational separation step.

5. The method according to claim 4, wherein the second solid stream is re-slurried and subjected to the third decantation or gravitational separation step for separating a third solid stream.

6. The method according to claim 1, wherein the method further comprises adding the flocculant additive to the hydrolyzed material before the decantation or gravitational separation step, to a re-slurried solid stream of the decantation or gravitational separation, or in connection with the re-slurrying.

7. The method according to claim 1, wherein the method further comprises feeding the flocculant additive to a feed of a pump located before the decantation or gravitational separation step, to a feeding line between the pump and the decantation or gravitational separation step, to the decantation or gravitational separation step, or just before a mixer or an inline mixer located before the decanter or gravitational separator.

8. The method according to claim 1, wherein the flocculant additive is a cationic polymer flocculant.

9. The method according to claim 1, wherein the method further comprises separating a residual solid stream from the liquid stream after the decantation or gravitational separation step.

10. The method according to claim 9, wherein the method further comprises feeding the flocculant additive to the liquid stream before separating the residual solid stream from the liquid stream.

11. The method according to claim 1, wherein the method further comprises:
iii) separating a solid product fraction from a re-slurried solid stream in a filtration separation step after the decantation or gravitational separation step.

12. The method according to of claim 11, wherein the method further comprises re-slurring the solid stream in a re-slurring step, adding a solid additive to the solid stream in the re-slurring, and separating the solid product fraction from the re-slurried solid stream in the filtration step.

13. The method according to claim 1, wherein the decantation or gravitational separation step comprises one or more decanter or gravitational separator.

14. The method according to claim 11, wherein the method further comprises recirculating at least a part of the liquid stream from the decantation or gravitational separation step or from the filtration step to the enzymatic hydrolysis and/or to a re-slurring step.

15. A process arrangement for improving a solid-liquid separation of solids from a hydrolyzed material formed in an enzymatic hydrolysis of lignocellulosic material, wherein the process arrangement comprises:
at least one feeder for feeding a flocculant additive to the hydrolyzed material or to a stream separated from the hydrolyzed material to form a mixture; and
at least one decanter or gravitational separator for separating a solid stream and a liquid stream from the mixture.

16. The process arrangement according to claim 15, wherein the feeder is arranged to feed the flocculant additive to a feed of a pump located before the decanter or gravitational separator, to a feeding line between the pump and the decanter or gravitational separator, to the decanter or gravitational separator, or just before a mixer or an inline mixer located before the decanter or gravitational separator.

17. The process arrangement according to claim 15 or 16, wherein the process arrangement further comprises at least one re-slurring tank for re-slurring the solid stream of the decanter or gravitational separator.

18. The process arrangement according to claim 17, wherein the feeder is arranged to feed the flocculant additive to the re-slurring tank.

19. The process arrangement according to claim 15, wherein the process arrangement further comprises at least one filtration device for separating a solid product fraction from a re-slurried solid stream of the decanter or gravitational separator.

20. The process arrangement according to claim 19, wherein the process arrangement further comprises a second feeder for feeding a solid additive to the re-slurried solid stream of the decanter or gravitational separator before the filtration device.

21. The process arrangement according to claim 15, wherein the process arrangement further comprises at least one separator for separating a residual solid stream from the liquid stream after the decanter or gravitational separator.

22. The process arrangement according to claim 21, wherein the feeder is arranged to feed the flocculant additive to the liquid stream before the separator in which the residual solid stream is separated from the liquid stream.

23. The process arrangement according to claim 15, wherein the process arrangement further comprises at least one recirculating device for recirculating at least a part of the liquid stream from the decanter or gravitational separator or from a filtration device to the enzymatic hydrolysis and/or to a re-slurring tank.

24. The process arrangement according to claim 15, wherein the process arrangement further comprises one or more enzymatic hydrolysis equipment.

25. The solid product obtainable by the method according to claim 1, wherein the solid product contains at least lignin.
